(19) United States
(12) Patent Application Publication
(10) Pub. No.: US 2009/0298045 A1
(43) Pub. Date: Dec. 3, 2009

(54) Method For Selectively Expanding, Selecting And Enriching Stem/Progenitor Cell Populations

(75) Inventors: Avraham J. Treves, Mevaseret Zion (IL); Arnon Nagler, Jerusalem (IL); Hanan Galsky, Jerusalem (IL); Iris Bar, Modiin (IL)

Correspondence Address: DR. MARK M. FRIEDMAN;C/O BILL POLKINGHORN - DISCOVERY DISPATCH, 9003 FLORIN WAY, UPPER MARLBORO, MD, 20772, US

(73) Assignee: Tel Hashomer-Medical Research, Infrastructure And Services Ltd., Ramat Gan (IL)

(21) Appl. No.: 11/568,702

(22) PCT Filed: May 5, 2005
(86) PCT No.: PCT/IL05/00475

§ 371 Date: Jul. 5, 2009

Related U.S. Application Data
(60) Provisional application No. 60/568,248, filed on May 6, 2004.

(51) Int. Cl. A01N 1/00; C12N 5/06

(52) U.S. Cl. 435/2; 435/375

(57) ABSTRACT

A method of producing stem/progenitor cells from human or animal origin. A population, from an embryonic, fetal or adult source, preferably from bone marrow, blood, fat, muscle, heart, intestine, kidney, liver, lung, pancreas, skin or neural tissues, that includes stem/progenitor cells, is treated with one or more first cytostatic or cytotoxic agents to which the stem/progenitor cells are less sensitive than the other cells of the population. Preferably, the agent(s) selectively deplete(s) from the population cells that are negative with respect to expressing a transporter gene of the first agent(s) while sparing cells that are positive with respect to expressing that gene. Preferably, the population also is treated with one or more cytokines and/or growth factors.

### FIELD AND BACKGROUND OF THE INVENTION

The present invention relates to production of stem cells and, more particularly, to a method of ex-vivo enrichment of stem cells in a population of cells.

### 1. THE NEED

Successful ex vivo expansion of stem and progenitor cells could be exploited for a variety of clinical applications. For example, such applications include increasing the number of stem cells available for genetic modification and bone mar-row transplantation as well as the generation of large quantities of immunologically reactive cells (T cells, natural killer cells, dendritic cells, and others) for adoptive immunotherapeutic purposes (Devine 2003). Studies to date have not addressed whether true hematopoietic stem cells with long-term repopulating potential can be expanded in culture, and transfused to shorten the period of pancytopenia and improve the overall rate of engraftment. In the light of this, there is a critical need in the invented method for ex vivo expansion of enriched stem cell populations.

The invented process will be suitable for both autologous and allogeneic transplantations as it will result in a reduction of the volumes of transplants to be cryopreserved. In addition, a smaller scale immunoselection technique, requiring less monoclonal antibody can be used for final purification after stem cells have been enriched by the invented procedure.

The invented method may be also useful in the ex vivo expansion of the following stem cells: bone marrow, peripheral blood, umbilical cord blood, myoblasts, cardiomyoblasts, hepatic stem cells, neural stem cells, mesenchymal stem cells, endothelial stem cells, embryonic stem cells, fetal stem cells or any other type of pluripotent stem cells that express this property (i.e., relatively higher resistance to cytostatic/cytotoxic agents). For example, in treatment of cardiac insufficiency ex vivo expanded stem cells could be accessible as single cell types with ideal properties as donor cells (Perry 2003).

In addition, bone marrow and umbilical cord blood derived stem cells have been used in pre-clinical models of brain injury, directed to differentiate into neural phenotypes, and have been related to functional recovery after engraftment in central nervous system (CNS) injury models (Newman 2003). Therefore, the suggested technique for stem cell enrichment may be combined also in cell-based transplantation therapies for neurodegenerative disorders.

Its use will also be advantageous in stem cells- and cancer-research and clinical applications due to its lower cost as compared to stem cells enrichment techniques based on antibodies.

### 2. BACKGROUND

### 2.1 Stem Cells Transplantation

Hematopoietic progenitor cells can be mobilized from the bone marrow (BM) to the blood by a wide variety of stimuli, including hematopoietic growth factors, chemotherapy, and chemokines. Moreover, it was observed that the mobilizing effect of chemotherapy can be enhanced by in vivo administration of hematopoietic growth factors, such as granulocyte colony-stimulating factor (G-CSF). Increasingly, mobilized peripheral blood (PB) hematopoietic progenitor cells instead of BM hematopoietic progenitor cells have been used to reconstitute hematopoiesis after myeloablative therapy because of their reduced engraftment times and relative ease of collection (Thomas 2002). In allogeneic PB stem cells transplantation compared with allogeneic BM transplantation, the incidence and frequency of graft-versus-host (GVHD) is of concern because high number of T lymphocytes are infused in allogeneic PB stem cells transplantation. The incidence and severity of acute GVHD are not increased but chronic GVHD is higher in allogeneic PB stem cells transplantation compared with allogeneic BM transplantation (Kasai 2002).

Umbilical cord blood (UCB) has been rapidly established as an alternative source of stem cells to BM for allogeneic-related and unrelated hematopoietic transplantation. The main advantage of UCB transplantation is the relative ease of procurement and the lower-than-anticipated risk of severe acute GVHD (Koh 2004). The use of reduced-intensity or nonmyeloablative preparative regimens to allow engraftment of UCB broadens the scope of patients who may benefit from allogeneic immunotherapy, including elderly and medically infirm patients with no matched sibling donor. To date, about 200,000 UCB units are available for transplantation and more than 3500 UCB transplants have been performed, mostly in children, for the treatment of a variety of malignant and nonmalignant conditions (Benito 2004). The major disadvantage of UCB is the low yield of stem cells, resulting in higher graft failure rates and slower time to engraftment compared to BM transplantation. A rational approach thus involves ex vivo expansion of UCB-derived hematopoietic precursors (Cohen 2004).

Therefore, our novel method of stem cell expansion will be demonstrated on UCB derived hematopoietic stem cells, although it may be also suitable for non-hematopoietic stem cells, e.g., mesenchymal stem cells that are an attractive therapeutic tool for cell transplantation and tissue engineering as well (Iris 2003).

### 2.2 Multidrug Resistant −1 (MDR1) and ABCG2 Gene Expression and Resistance to Cytostatic and/or Cytotoxic Agents

Resistance to various chemically different natural product, anti-cancer drugs (multidrug resistance, or MDR) results from over-expression of one or more ATP-dependent efflux transporters and subsequent decreased drug accumulation. The first of these to be identified was P-glycoprotein (Pgp), the product of the human MDR1 (ABCB1) gene, localized to chromosome 7q21. Pgp is a member of the large ATP-binding cassette (ABC) family of proteins (Ambudkar 2003, Findling-Kagan 2005, Galski 2003, Sauna 2001), ABCG2 (BCRP1) is another member of the ABC family of cell surface transport proteins, located on chromosomal locus 4q22 (Abbott 2003). The substrate profiles of Pgp (MDR1) and ABCG2 transporters include various cytostatic agents and antineoplastic drugs

### 2.3 Stem Cells have Relative High Expression Level of MDR1 and ABCG2

There are several indications that stem cells express MDR1 in relatively high levels. For example Bertolini 1997 showed that after drug exposure most of the peripheral blood progenitor cells displayed a CD34⁺, CD38⁻, MDR1⁺, Rhodamine 123 (Rh123) low and Hoechst 33342 (Ho) low phenotype, and as few as 180 of these drug-resistant cells were able to generate a stable multilineage human hematopoiesis in sublethally irradiated immunodeficient mice. Recently Uchida 2004 demonstrated that activation of adult hematopoietic stem cells (HSC) in vivo following 5-fluorouracil treatment, or in vitro with cytokines, induces variable losses in Rh123 and Ho efflux activities. Moreover, HSC from MDR1a/1b(−/−) mice show a dramatic decrease in Rh123 efflux ability. The supravital dyes Rh123 and Ho are powerful probes for the characterization, resolution, isolation, and purification of primitive HSC. The fidelity of Rh123 and Ho as stem cell probes resides in their individual and combined ability to hierarchically order the HSC on the basis of their probability of cycling by probing individual traits that define the quiescent state, and by so exploiting the overlapping activity of transmembrane efflux pumps belonging to the ABC transporter superfamily, including MDR1, MRP1, and BCRP1/ABCG2, for which they are preferential substrates (Bertoncello 2004). ABCG2 expression was shown within a more primitive subpopulation of cells than MDR1 (Zhou 2001).

Recent studied also indicate that non-hematopoietic stem and/or progenitor cells express MDR1 in relatively high levels. Study of Fiaccavento 2005 demonstrated the presence of an increased number of c-kit positive, MDR-positive, and Sca-1-positive stem cells within the myocardium of hereditary delta-SG null hamsters, a spontaneously occurring model of hypertrophic cardiomyopathy. Moreover, hepatic progenitor cells of rats treated with 2-acetylaminofluorene followed by partial hepatectomy express high levels of active multidrug resistance protein 1 (MRP1) and MRP3, as determined by real time detection RT-PCR (Ros 2003). MRP1, MRP2 and MRP3 also belong to the ABC transporter family.

### 2.4 Surface Antigens Expression of HSC

Prominin, also termed CD133 (AC133), is a highly conserved antigen expressed on HSC associated with mobility and primitive function. Koehl 2002 reported for the first time a successful transplantation with a CD133 positive, selected graft. The fact that CD133⁺ hematopoietic progenitors can give rise to an adherent population which is CD133⁻ and CD34⁻ and that these cells can again give rise to a CD133⁺CD34⁻ stem cell population with high NOD/SCID engraftment potential (Handgretinger 2003), indicates that CD133 might be an earlier marker of hematopoietic precursors than CD34. Dimitriou 2003 observed a more effective proliferation of the CD34⁺ population than the CD133⁺ population, while the CD133⁺ cell fraction retained and expanded more immature elements. Therefore, they concluded that CD133⁺ and CD34⁺ expanded UBC cells could potentially be used in combination to overcome the shortcomings of cord blood transplantation in older children and adults (Dimitriou 2003). Recently, Forraz 2004 isolated a discrete lineage-negative (Lin⁻) cell population that maintained/expanded more primitive hematopoietic stem and progenitor cell than CD133⁺ cells but underwent slow proliferation. Lately Wagner 2004 showed that the slow dividing fraction of hematopoitic progenitor cells associated with primitive function and self-renewal is characterized by a highest expression level of CD133 and MDR1 genes as detected by microarray analysis. This study strengthens our rational that treatment with cytostatic and/or cytotoxic agents could selectively affect mature and differentiated cells (MDR1/ABCG2 negative) while supporting the growth of stem cells (MDR1/ABCG2 positive).

### 2.5 Current Techniques for Stem Cell Enrichment

Several immunological techniques have been developed for the isolation of stem cells. In most of the available methods, CD34 or CD133 monoclonal antibodies (mAbs), which react specifically with hematopoietic stem cells, are being used for the isolation of progenitor cells.

One major disadvantage of the available techniques is the large amount of mAbs needed, which makes these techniques very expensive. A second disadvantage is the need of some technique to release the cells, which may result in damage to the progenitor cells. The use of affinity columns to separate stem cells is difficult when frozen cells are used (e.g., frozen cord blood units), mainly due to aggregation of the thawed cells. Therefore, a process leading to substantial enrichment of stem cells by depletion of non-stem cells, without the constant use of mAbs or affinity columns would be a considerable advantage.

Several methods have also been reported for ex vivo expansion of stem cells, but these methods always require a step of immuno-selective enrichment of the stem cells before and/or during expansion (Gammaitoni 2003, Petzer 1997, Peled 2002).

### SUMMARY OF THE INVENTION

The present invention is a procedure for enrichment and selective expansion of stem cells and/or progenitor cells based on selective killing (or growth arrest) of MDR1 negative non-progenitor cells by treatment with a combination of a cytostatic and/or cytotoxic reagent together with a cocktail of growth factors. These conditions will selectively kill mature and differentiated cells (MDR1/ABCG2/MRP negative) while supporting the growth of stem cells (MDR1/ABCG2/MRP positive).

According to the present invention there is provided a method of producing stem cells, including the steps of: (a) providing a population that includes the stem cells; and (b) treating the population with at least one first agent, selected from the group consisting of cytostatic agents and cytotoxic agents, to which the stem cells are less sensitive than other cells in the population.

The population may be obtained from an embryonic source, from a fetal source or from an adult source.

Preferably, the population is obtained from bone marrow, blood (e.g., peripheral blood or umbilical cord blood), fat, muscle, skin or any other tissue as neural tissue or tissue from internal organs such as the heart, the intestine, a kidney, the liver, a lung or the pancreas.

Preferably, the first agent(s) selectively deplete(s) cells that are negative with respect to expressing a transporter gene of the first agent(s) while tending to spare cells that are positive with respect to expressing that gene. Examples of such transporter genes include members of the ABC transporter gene family such as MDR1, ABCG2, or members of the MRP transporter gene family such as MRP1, MRP2 and MRP3.

Preferably, the method also includes the step of treating the population with one or more cytokines or growth factors.

Preferably, the treating with the first agent(s) is effected ex-vivo.

The scope of the term “stem cells” as used in the appended claims includes both stem cells and progenitor cells.

### BRIEF DESCRIPTION OF THE DRAWINGS

The invention is herein described by way of example only, with reference to the accompanying drawings, wherein:

FIG. 1 shows expression (RT-PCR) of (A) MDR1− and (B) β-actin-RNA in UCB-derived CD133+ cells relative to CD133− and mononuclear cells (MNC). RNA was extracted from: 1, KB-8-5 cell line (MDR1 positive control); 2, fresh enriched CD133− positive cell fraction; 3, CD133 negative-cell fraction; 4, MNC isolated by density gradient centrifugation.

FIG. 2 shows flow cytometry (FACS) analyses for Pgp (MDR1) expression of MNC and CD133+ enriched cells (CD133 positive fraction) isolated form fresh UCB. A representative analysis is shown.

FIG. 3 is a bar graph of dose-dependent enrichment of UCB-derived CD133+ cell-fraction by cytostatic agent addition to expansion medium. Representative results from flow cytometry analyses after 2 weeks expansion in culture are shown. Results are indicated as % of CD133 positive cells from total viable cells (7-AAD unstained cells).

### DESCRIPTION OF THE PREFERRED EMBODIMENTS

The present invention is of a process for the preparation of enriched populations of stem/progenitor cells for various cell therapy applications, without the continuous use of xenogeneic antibodies directed against cell surface molecules, affinity columns or density so gradients. The invention includes a method for enrichment and expansion of stem/progenitor cells based on their relatively high expression levels of multidrug resistant −1 (MDR1, also termed ABCB1) gene and/or ABCG2 gene and/or MRP gene, as compared to more differentiated progenitor and mature cells. The invention relates to expansion of stem/progenitor cells ex-vivo (in the presence of cytokines and growth factors) using selective killing of MDR1 and/or ABCG2 and/or MRP low/negative cells by cytostatic and/or cytotoxic agents while avoiding the killing of MDR1 and/or ABCG2 and/or MRP positive stem/progenitor cells. The principles and operation of stem/progenitor cell enrichment according to the present invention may be better understood with reference to the drawings and the accompanying description.

### 3. EXAMPLE

### 3.1 Methods

### 3.1.1 Cord Blood Samples

Cells were obtained from human umbilical cord blood (UCB) after normal fall-term delivery. The cells were layered on a Ficoll-Hypaque gradient (1.077 g/ml Sigma), and centrifuged at 500 g for 30 minutes. The mononuclear cells in the interface layer were collected and washed three times in phosphate-buffered saline (PBS; Biological Industries) containing 0.5% Bovine serum albumin (BSA). To purify the CD133⁺ cells, the mononuclear cell fraction was subjected to two cycles of immunomagnetic bead separation using a MiniMACS CD133 progenitor cell isolation kit (Miltenyi Biotec,), according to the manufacturer's recommendations.

The purity of the CD133⁺ population thus obtained was evaluated by flow cytometry (see section 3.1.3).

3.1.2 Ex vivo Expansion

Purified CD133⁺ cells were cultured in 24-well culture plate at densities of 2-4×10⁴ cells in alpha-MEM medium (Biological Industries) supplemented with 10% fetal calf serum (Biological Industries) and the following human recombinant cytokines (Pepro Tech, Inc., Rocky Hill, N.J., USA): Trombopoietin, interleukin-6, FLT-3 ligand and Stem Cell Factor, at final concentration of 50 ng/ml each as well as interleukin-3 and IL2 at 20 ng/ml, in the absence or presence of colchicine in a concentration scale as indicated. Cells were expanded at 37° C. in a humidified atmosphere of 5% CO₂ in air.

Cultures were depopulated twice a week and a fresh medium in which growth factors and colchicine were added. At various time points, cells were counted after staining with trypan blue and harvested cells were used for enumeration of CD133⁺ cells following immunophenotype analysis.

### 3.1.3 Flow Cytometry for Analyses of Surface Antigens

The cells were washed with a PBS solution containing 1% BSA and stained (at 4° C. for 30 min) with fluorescein isothiocyanate (FITC)- or phycoerythrin (PE)-conjugated antibodies that are specifically described in the next sections. Cells were washed in the abovementioned buffer and analyzed by flow cytometer (Becman coulter). Cells were passed at a rate of up to 1000 cells/second, using a 488-nm argon laser beam as the light source for excitation.

Cells stained with FITC- and PE-conjugated isotype control antibodies were used to determine background fluorescence.

3.1.4 Determination of CD133⁺ Cell Subset after Expansion

The percentage of CD133⁺ cell fraction from total cells was determined by flow cytometry using. PE-anti-CD133⁺ antibody (Miltenyi Biotec). Fold enrichment was calculated by dividing the CD133⁺ cell percentage of culture growing with cytostatic agent by the CD133⁺ cell percentage of culture growing without cytostatic agent. Since cultures were depopulated twice a week, the culture volume was calculated by multiplying the actual volume with the number of passages. Fold expansion was calculated by dividing the CD133⁺ cell content of the culture by the initial number of cultured CD133⁺ cells.

### 3.1.5 Determination of Early Stem Cell Subsets

The percentages of early stem cell subsets were determined from the purified fresh and/or cultured CD133⁺ cell fraction. Cells were dually stained with PE anti-CD34 and FITC anti-CD38 (DAKO) for determination of CD34⁺ CD38⁻ cells by flow cytometry analyses with PE anti-CD133⁺ antibody (Miltenyi Biotec).

### 3.1.6 Determination of Pgp (MDR1) Surface Expression in Stem Cell Subsets

Cells were dually stained with PE anti-CD133⁺ antibody (Miltenyi Biotec) and with MRK16 antibody against Pgp and FITC secondary goat anti mouse antibody (Jackson). Stained cells were subjected to FACS analyses.

### 3.1.7 Preparation and Analysis of RNA

Total RNA was isolated using RNeasy Mini kit (Quiagen Sciences, Germantown, Md., USA), according to the manufacturer instructions. RNA expression was measured by RT-PCR cDNA was synthesized by reverse transcription system (Promega, Madison, Wis., USA), according to manufacturer instructions. The cDNA was used as a template for subsequent amplification of the human MDR1 gene transcript. Amplification of the β-actin gene was used as internal control.

RT-PCR was performed using the primers,

F: 5′-TTTACTGATAAAGAACTCTTA-3′

R: 5′-AACTGAAGTGAACATTTCTG-3′ for human MDR1, (product size 487 bp) and the primers

F: 5′-CCAAGGCCAACCGCGAGAAGATGAC-3′

R: 5′-AGGGTACATGGTGGTGCCCCGAGAC-3′ for β-actin (product size 589 bp).

Reaction mixtures contained the following: 0.5 μg cDNA, 25 pM of each primers and 2× ReddyMix PCR Master Mix (ABgene, Surrey, UK), which contains 1.25 U Thermoprime Plus DNA Polymerase, 75 mM Tris-HCl (pH 8.8), 20 mM (NH₄)₂SO₂, 1.5 mM MgCl₂, 0.01% Tween 20, 0.2 mM of each dNTP, precipitant and red dye for electrophoresis. Cycling parameters were: denaturation in 94° C. for 1 min, annealing in 61° C. for 1 min, and extension in 72° C. for 1 min. Samples were cycled using a Paltier Thermal Cycler, PTC-200, (MJ Research, Watertown, Mass., USA).

The RT-PCR, products were separated by 2% agarose gel electrophoresis and visualized under UV light using ethidium bromide staining. Gels were scanned and analyzed by EDAS 290 Electrophoresis Documentation and Analysis System Kodak).

### 3.2 Results

### 3.2.1 Selection and Characterization of Enriched Stem Cell Population

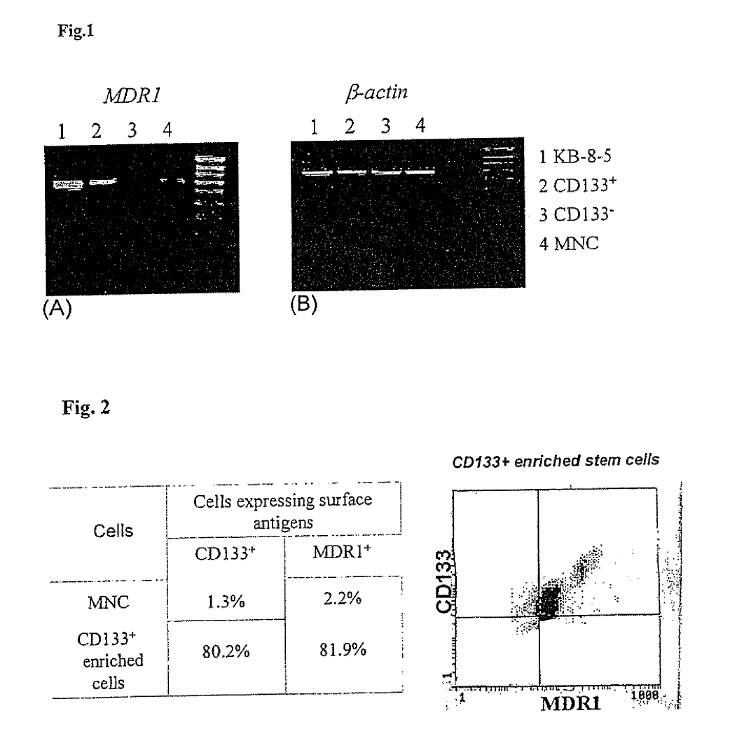

Enriched CD133⁺ stem cell population was prepared from human, UCB-derived MNC by CD133 immunomagnetic beads isolation kit. The MDR1 expression level of UCB-derived CD133⁺ cells in comparison to UCB-derived MNC and CD133⁻ cells was studied using RT-PCR and flow cytometry (FIGS. 1 and 2, respectively). As shown in FIG. 1A, MDR1 RNA expression level in UCB-derived CD133⁺ cells is significantly higher than its expression in both UCB-derived MNC and CD133⁻ cells. The expression level of the house-keeping gene, β-actin, was similar in all the tested sub-populations (FIG. 1B). The purity of CD133⁺ cells was measured by flow cytometry (FIG. 2). Results indicated that approximately 80% of the CD133 enriched cells were positive for CD133 and for Pgp (MDR1) antigens. Pgp expression in UCB derived CD133⁺ cells is significantly higher than its expression in MNC (81.9% v.s. 2.2%, respectively). Moreover, most of the UCB-derived CD133⁺ cells also express Pgp. These results indicate that CD133⁺ stem cells express higher level of MDR1 relatively to the other cells and, therefore, may be further selected by cytostatic agents to enrich their fraction.

### 3.2.2 Effect of Cytostatic Agent Treatment on Enrichment and Expansion of CD133⁺, Stem Cell Population

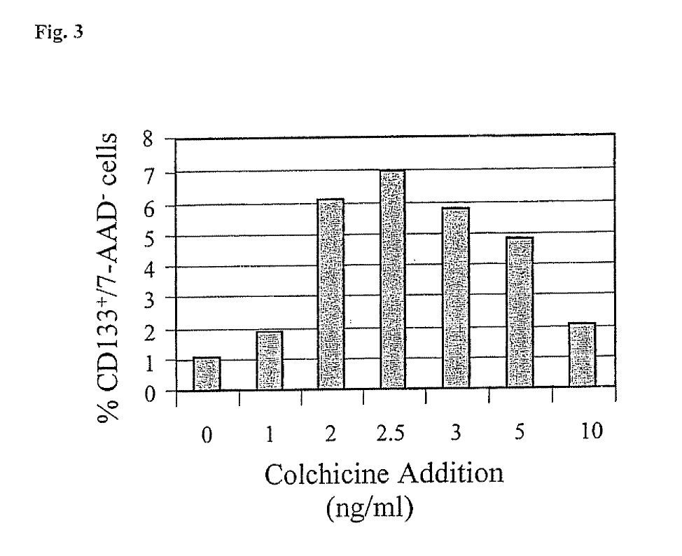

Purified UCB-derived CD133⁺ cells were cultured in cytokine-supplemented liquid medium in the continuous presence or absence of cytostatic agent (colchicine) in a concentration scale (FIG. 3). Flow cytometry analysis after 2 weeks expansion (of three independent experiments) demonstrated a dose-dependent enrichment of UCB-derived CD133⁺ cell-fraction by the cytostatic agent colchicine and revealed the optimal concentration of 2.5 ng/ml in which the highest CD133⁺ cells enrichment was achieved (FIG. 3).

Although intrinsic variability was demonstrated in the ex vivo expansion potential of CD133⁺ cells derived from different cord blood donors, the use of cytostatic agent substantially increased the renewal potential, resulting in prolongation of CD133⁺ cell enrichment and expansion (Table 1 and Table 3).

### 3.2.3 Effect of Cytostatic Agent Treatment on Early Progenitors

CD34⁺ hematopoietic progenitors comprise a heterogeneous population. The minority, CD34⁺CD38⁻ are lineage uncommitted progenitors, whereas the majority, CD34⁺CD38⁺ cells, are lineage committed cells.

To determine the effect of cytostatic agent on CD34⁺ CD38⁻ early stem cell sub-population, purified UCB derived CD133⁺ cells after 1 week in culture in the absence or presence of colchicine were analyzed by flow cytometry for CD34 and CD38 surface antigen expression. The results (summarized in table 2) indicated an increase in the percentage of CD34⁺CD38⁻ sub-population in cultures treated with cytostatic agent comparing to un-treated cultures. These results demonstrated that colchicine enabled preferential proliferation of early progenitor cells with CD133⁺ and CD34⁺38⁻ phenotype, resulting in the observed increased ex vivo expansion.

TABLE 1
Enrichment of CD133⁺ stem cell population in culture
in absence and presence of cytostatic agent.

| | CD133+ cell | CD133+ cell | |
|---|---|---|---|
| | population without | population with | |
| Experiment | cytostatic agent | cytostatic agent | Fold enrichment |
| Donor # 1 | 1% | 4% | 4 |
| Donor # 1 | 1% | 7% | 7 |
| Donor # 3 | 6% | 15% | 2.5 |
| Mean ± SD | 2.7 ± 2.9 | 8.7 ± 5.7 | 4.5 ± 2.3 |

Results obtained after expansion of CD133 positive cells for two weeks in the absence or presnece of cytostatic agent (colchicine) at 2.5 ng/ml. Results from three independent experiments of different UCB donors are shown.

TABLE 2
Enrichment of CD34⁺38⁻, early stem cell population
in cultrue in absence and presence of cytostatic agent.

| | CD34+38− cell | CD34+38− cell | |
|---|---|---|---|
| | population without | population with | |
| Experiment | cytostatic agent | cytostatic agent | Fold enrichment |
| Donor # 3 | 9% | 15% | 1.7 |
| Donor # 4 | 0.5% | 5% | 10 |

Results obtained after expansion of CD133 positive cells for one week in the absence or presence of cytostatic agent (colchicine) at 2.5 ng/ml.

TABLE 3
Enhanced stem cells expansion by cytostatic agent. Fresh, enriched
CD133⁺ stem cells from various donors were plated at densities
indicated at T₀. Counts of total cells and CD133⁺ cells are
shown after 2 weeks of ex-vivo expansion in the absence or presence of
cytostatic agent (colchicine at 2.5 ng/ml).
Fold expansion is shown in parentheses.

| | | T_{(2 weeks)} | | | |
|---|---|---|---|---|---|
| | T₀ | Without colchicine | | With 2.5 ng · ml colchicine | |
| | CD133+ | | CD133+ | | CD133+ |
| Exp. | cells | Total cells | cells | Total cells | cells |
| 1 | 0.4 × 10³ | 270 × 10³ | 2.7 × 10³ | 135 × 10³ | 5.4 × 10³ |
| | | | (X 6.5) | | (X 13.5) |
| 2 | 0.2 × 10³ | 135 × 10³ | 1.3 × 10³ | 68 × 10³ | 4.7 × 10³ |
| | | | (X 6.5) | | (X 23.5) |
| 3 | 0.4 × 10³ | 81 × 10³ | 4.9 × 10³ | 27 × 10³ | 4.1 × 10³ |
| | | | (X 12.3) | | (X 10.2) |

Based on the abovementioned results, the invention provides a process for the selection, enrichment and expansion of stem/progenitor cells subsets that are commonly a constituent of umbilical cord blood, peripheral blood, and bone marrow. The process could be also suitable for selective enrichment and expansion of other types of stem/progenitor cells that predominantly express Pgp (MDR1).

While the invention has been described with respect to a limited number of embodiments, it will be appreciated that many variations, modifications and other applications of the invention may be made.

### REFERENCES

Abbott B L. ABCG2 (BCRP) expression in normal and malignant hematopoietic cells. Hematol Oncol. 2003, 21:115-30. Review.
Ambudkar S V, Kimchi-Sarfaty C, Sauna Z E, Gottesman M M. P-glycoprotein: from genomics to mechanism. Oncogene. 2003, 22:7468-85. Review.
Benito A I, Diaz M A, Gonzalez-Vicent M, Sevilla J, Madero L. Hematopoietic stem cell transplantation using umbilical cord blood progenitors: review of current clinical results. Bone Marrow Transplant. 2004, 33:675-90.
Bertolini F, Battaglia M, Lanza A, Gibelli N, Palermo B, Pavesi L, Caprotti M, Robustelli della Cuna G. Multilineage long-term engraftment potential of drug-resistant hematopoietic progenitors. Blood. 1997, 90:3027-36.
Bertoncello I, Williams B. Hematopoietic stem cell characterization by hoechst 33342 and rhodamine 123 staining. Methods Mol Biol. 2004; 263:181-200.
Chopp M and Y Li. Treatment of neural injury with marrow stromal cells. Lancet Neurol. 2002 1:92-100.
Cohen Y, Nagler A. Cord blood biology and transplantation. Isr Med Assoc J. 2004, 6:39-46. Review.
Devine S M, Lazarus H M, Emerson S G. Clinical application of hematopoietic progenitor cell expansion: current status and future prospects. Bone Marrow Transplant. 2003, 31:241-52. Review.
Dimitriou H, Vorgia P, Stiakaki E, Mavroudis D, Markaki E A, Koumantakis E, Kalmanti M. In vitro proliferative and differentiating characteristics of CD133(+) and CD34(+) cord blood cells in the presence of thrombopoietin (TPO) or erythropoietin (EPO). Potential implications for hematopoietic cell transplantation. Leuk Res. 2003, 27:1143-51.
Handgretinger R, Gordon P R, Leimig T, Chen X, Buhring H J, Niethammer D, Kuci S. Biology and plasticity of CD133⁺ hematopoietic stem cells. Ann N Y Acad Sci. 2003, 996:141-51.
Fiaccavento R, Carotenuto F, Minieri M, Fantini C, Forte G, Carbone A, Carosella L, Bei R, Masuelli L, Palumbo C, Modesti A, Prat M, Di Nardo P. Stem cell activation sustains hereditary hypertrophy in hamster cardiomyopathy. J Pathol. 2005, 205:397-407.
Findling-Kagan S, Sivan H, Ostrovsky O, Nagler A, Galski H. Establishment and characterization of new cellular lymphoma model expressing transgenic human MDR1. Leuk Res. 2005, 29:407-14.
Forraz N, Pettengell R, McGuckin C P. Characterization of a lineage-negative stem-progenitor cell population optimized for ex vivo expansion and enriched for LTC-IC. Stem Cells. 2004, 22:100-8.
Galski H, Nagler A. Novel inhibitors of multidrug resistance (MDR): Evaluation studies for future clinical applications. Ann Proc Eur Hematol 2003, 7:129
Gammaitoni L, Bruno S, Sanavio F, Gunetti M, Kollet O, Cavalloni G, Falda M, Fagioli F, Lapidot T, Aglietta M, Piacibello W. Ex vivo expansion of human adult stem cells capable of primary and secondary hemopoietic reconstitution. Exp Hematol. 2003, 31:261-70.
Iris B, Zilberman Y, Zeira E, Galun E, Honigman A, Turgeman G, Clemens T, Gazit Z, Gazit D. Molecular imaging of the skeleton: quantitative real-time bioluminescence monitoring gene expression in bone repair and development. J Bone Miner Res. 2003, 18:570-8.
Kasai M, Kiyama Y, Kawamura A. Application of peripheral blood stem cells (PBSC) mobilized by recombinant human granulocyte colony stimulating factor for allogeneic PBSC transplantation and the comparison of allogeneic PBSC transplantation and bone marrow transplantation. Transfus Apheresis Sci. 2002, 26-121-7. Review,
Koh L P, Chao N J. Umbilical cord blood transplantation in adults using myeloablative and nonmyeloablative preparative regimens. Biol Blood Marrow Transplant 2004, 10:1-22.
Koehl U, Zimmermann S, Esser R, Sorensen J, Gruttner H P, Duchscherer M, Seifried E, Klingebiel T, Schwabe D. Autologous transplantation of CD133 selected hematopoietic progenitor cells in a pediatric patient with relapsed leukemia. Bone Marrow Transplant. 2002, 29:927-30.
Newman M B, Davis C D, Kuzmin-Nichols N, Sanberg P R. Human umbilical cord blood (HUCB) cells for central nervous system repair. Neurotox Res. 2003, 5:355-68. Review.
Peled T, Landau E, Prus E, Treves A J, Fibach E. Cellular copper content modulates differentiation and self-renewal in cultures of cord-blood derived CD34⁺ cells. Brit. J. Haematol. 2002, 116:655-661
Perry T E, Roth S J. Cardiovascular tissue engineering: constructing living tissue cardiac valves and blood vessels using bone marrow, umbilical cord blood, and peripheral blood cells. J Cardiovasc Nurs. 2003, 18:30-7. Review.
Petzer A L, Eaves C J, Barnett M J, Eaves A C. Selective expansion of primitive normal hematopoietic cells in cytokine-supplemented cultures of purified cells from patients with chronic myeloid leukemia. Blood, 1997, 90:64-9
Ros J E, Roskams T A, Geuken M, Havinga R, Splinter P L, Petersen B E, LaRusso N F, van der Kolk D M, Kuipers F, Faber K N, Muller M, Jansen P L. ATP binding cassette transporter gene expression in rat liver progenitor cells. 2003, Gut. 52:1060-7,
Sauna Z E, Smith M M, Muller M, Kerr K M, Ambudkar S V. The mechanism of action of multidrug-resistance-linked P-glycoprotein. J Bioenerg Biomembr. 2001, 33:481-91. Review.
Thomas J, Liu F, Link D C. Mechanisms of mobilization of hematopoietic progenitors with granulocyte colony-stimulating factor. Curr Opin Hematol. 2002, 9:183-9. Review.
Uchida N, Dykstra B, Lyons K, Leung F, Kristiansen M, Eaves C. ABC transporter activities of murine hematopoietic stem cells vary according to their developmental and activation status. 2004, Blood 103:4487-95.
Wagner W, Ansorge A, Wirkner U, Eckstein V, Schwager C, Blake J, Miesala K, Selig J, Saffrich R, Ansorge W, Ho A D. Molecular evidence for stem cell function of the slow-dividing fraction among human hematopoietic progenitor cells by genome-wide analysis. Blood. 2004, 104:675-86.
Zhou S, Schuetz J D, Bunting K D, Colapietro A M, Sampath J, Morris J J, Lagutina I, Grosveld G C, Osawa M, Nakauchi R, Sorrentino B P. The ABC transporter Bcrp1/ABCG2 is expressed in a wide variety of stem cells and is a molecular determinant of the side-population phenotype. Nat Med. 2001, 7:1028-34.

What is claimed is:

1. A method for producing a population highly enriched for non-differentiated, stem/progenitor cells comprising:
(a) providing a population that includes the stem/progenitor cells;
b) treating said population with at least one first agent to which non-differentiated, stem/progenitor cells are less sensitive than are other cells in said population, said first agent selected from the group consisting of cytostatic agents and cytotoxic agents, said first agent inhibiting the proliferation or maturation of those mature and differentiated non-progenitor cells that are MDR1 low/negative or ABCG2 low/negative or MRP low/negative, thereby increasing the proportion of non-differentiated, stem/progenitor cells in the population;
(c) enriching the population by selecting and eliminating from the population those mature and differentiated non-progenitor cells that are MDR1 low/negative or ABCG2 low/negative or MRP low/negative, such that those non-differentiated, stem/progenitor cells that express an ABC transporter gene are selected to produce a population enriched for non-differentiated, stem/progenitor cells; and
(d) recovering an enriched, non-differentiated, stem/progenitor cell population.

2. The method of claim 1, wherein said population is obtained from an embryonic source.

3. The method of claim 1, wherein said population is obtained from a fetal source.

4. The method of claim 1, wherein said population is obtained from an adult source.

5. The method of claim 1, wherein said population is obtained from a source selected from the group consisting of bone marrow, blood, fat, muscle, skin, tissues of an internal organ and neural tissues.

6. The method of claim 5, wherein said blood is peripheral blood.

7. The method of claim 5, wherein said blood is umbilical cord blood.

8. The method of claim 5, wherein said internal organ is selected from the group consisting of heart, intestine, kidney, liver, lung and pancreas.

9. The method of claim 1 wherein said ABC transporter gene is selected from the group consisting of MDR1 (ABCB1), ABCG2, MRP1, MRP2 and MRP3.

10. The method of claim 1 further comprising, at the same time as step (b), the step of expansion of stem/progenitor cells by treating said population with at least one second agent selected from the group consisting of cytokines, growth factors, and combinations thereof.

11. The method of claim 1, wherein said treating is effected ex-vivo.

12. A method for performing ex vivo expansion of a stem/progenitor cell population having a phenotype selected from the group consisting of at least one of CD133⁺ and CD34⁺38⁻ phenotype in a population of cells, comprising the steps of:
(a) providing a population of cells that includes stem/progenitor cells;
(b) culturing said population ex-vivo with culture medium comprising at least one agent selected from the group consisting of cytostatic agents and cytotoxic agents to which stem/progenitor cells having CD133⁺ and/or CD34⁺38⁻ phenotype are less sensitive than are other cells in said population, thereby increasing the proportion of stem/progenitor cells having CD133⁺ and CD34⁺38⁻ phenotype in the population, while at the same time, substantially eliminating, or at least inhibiting proliferation or maturation of mature and differentiated non-progenitor cells;
(c) enriching the population of the CD133⁺ and/or CD34⁺38⁻ stem/progenitor cells by eliminating non-CD133⁺ and/or CD34⁺38⁻ expressing cells thereby expanding the stem/progenitor cell population having CD133⁺ and/or CD34⁺38⁻ phenotype; and
(c) harvesting said cultured stem/progenitor cell population having CD133⁺ and/or CD34⁺38⁻ phenotype.

13. The method of claim 12 wherein said culture medium further comprises at least one agent selected from the group consisting of cytokines, growth factors, and combinations thereof.

14. A method for the ex-vivo expansion of multipotential cells in a population of cells, comprising culturing the population in an incubation medium comprising at least one inhibitor of mature and differentiated non-progenitor cells, said inhibitor being present in amounts effective to produce a composition substantially enriched in a subpopulation of stem/progenitor cells as compared to expansion in the absence of said inhibitor.

15. The method of claim 14 wherein said at least one inhibitor is selected from the group consisting of cytostatic agents and cytotoxic agents.

16. The method of claim 14 wherein an agent selected from the group consisting of cytokines, growth factors, and combinations thereof is added to the incubation medium concurrently with said at least one inhibitor.

17. A pharmaceutical composition comprising a therapeutically effective amount of the cell population of claim 1 and a pharmaceutically acceptable carrier.

18. A method of treating a patient comprising administering to said patient a composition of claim 1.

19. A pharmaceutical composition comprising a therapeutically effective amount of the cell population of claim 14 and a pharmaceutically acceptable carrier.

20. A method of treating a patient comprising administering to said patient a composition of claim 14.
